(19) United States
(12) Patent Application Publication
(10) Pub. No.: US 2015/0190072 A1
(43) Pub. Date: Jul. 9, 2015

(54) SYSTEMS AND METHODS FOR DISPLAYING AND INTERACTING WITH DATA FROM AN ACTIVITY MONITORING DEVICE

(71) Applicant: JayBird LLC, Salt Lake City, UT (US)

(72) Inventor: Judd ARMSTRONG, Salt Lake City, UT (US)

(73) Assignee: JayBird LLC, Salt Lake City, UT (US)

(21) Appl. No.: 14/591,652

(22) Filed: Jan. 7, 2015

Related U.S. Application Data
(63) Continuation-in-part of application No. 29/513,066, filed on Dec. 24, 2014.
(60) Provisional application No. 61/924,486, filed on Jan. 7, 2014.

(51) Int. Cl. A61B 5/11; A61B 5/00; A61B 5/0205

(52) U.S. Cl. CPC A61B 5/1118; A61B 5/0205; A61B 5/742; A61B 5/6824; A61B 5/6826; A61B 5/7275; A61B 5/7282; A61B 5/4809; A61B 5/0022; A61B 5/02405

(57) ABSTRACT

Methods and systems for displaying and interacting with data from an activity monitoring device are disclosed. In one implementation, a system may include an activity monitoring device including a plurality of biosensors electrically coupled to a circuit board and configured to measure biological information of a user of the activity monitoring device; and a computing device communicatively coupled to the activity monitoring device. The computing device may include a display; one or more processors; and one or more non-transitory computer-readable mediums operatively coupled to at least one of the one or more processors and having instructions stored thereon that, when executed by at least one of the one or more processors, cause: at least one of the one or more processors to process the biological information measured by the activity monitoring device, and the display to display an activity display and a sleep display based on the processed biological information.

### CROSS-REFERENCE TO RELATED APPLICATIONS

This application claims the benefit of U.S. Provisional Application No. 61/924,486 filed on Jan. 7, 2014, which is incorporated herein by reference in its entirety, and is a continuation-in-part of U.S. Design patent application No. 29/513,066 filed on Dec. 24, 2014, which is incorporated herein by reference in its entirety.

### TECHNICAL FIELD

The present disclosure relates generally to activity monitoring devices, and more particularly, some embodiments relate to systems and methods for displaying and interacting with data from an activity monitoring device.

### BRIEF SUMMARY OF THE DISCLOSURE

Systems and methods are provided in various embodiments for displaying and interacting with data from an activity monitoring device. In one embodiment, a system includes an activity monitoring device including a plurality of biosensors electrically coupled to a circuit board and configured to measure biological information of a user of the activity monitoring device, and a computing device communicatively coupled to the activity monitoring device. The computing device may include a display; one or more processors; and one or more non-transitory computer-readable mediums operatively coupled to at least one of the one or more processors and having instructions stored thereon that, when executed by at least one of the one or more processors, cause: at least one of the one or more processors to process the biological information measured by the activity monitoring device, and the display to display an activity display and a sleep display based on the processed biological information. In an implementation of this embodiment, the wristband includes a cavity notched on a radially inward side of the wristband that is shaped to substantially match a profile of the electronic capsule such that the electronic capsule is form-fit in place when positioned in the cavity.

In accordance with another embodiment of the technology disclosed herein, a method includes: collecting biological information of a user using one or more biosensors on an activity monitoring device; processing the biological information; displaying on a device display an activity display based on the processed biological information; displaying on the device display a sleep display based on the processed biological information; and displaying on the device display a recommendation and fatigue level display based on the processed biological information.

In an implementation of this embodiment, the processing operation and the displaying operations are performed when one or more processors of the device execute an activity tracking application stored on a non-transitory computer-readable medium of the device. In further implementations, displaying the activity display includes displaying at least three of a display navigation area, activity icons, an activity goal section, a live activity chart, and an activity timeline.

In accordance with yet another embodiment of the technology disclosed herein, a method includes displaying to a user an instruction to place a finger on a heart rate sensor of an activity monitoring device; measuring the user's heart rate variability (HRV) for a predetermined amount of time using the heart rate sensor; based on the user's measured HRV, determining a fatigue level and recommended amount of activity for the user for the current day; and displaying to the user the fatigue level and recommended amount of activity for the day. In implementations of this embodiment, the displayed fatigue level, and the displayed recommend amount of activity are displayed on a mobile device communicatively coupled to the activity monitoring device.

Other features and aspects of the disclosed method and system will become apparent from the following detailed description, taken in conjunction with the accompanying drawings, which illustrate, by way of example, the features in accordance with embodiments of the disclosure. The summary is not intended to limit the scope of the claimed disclosure, which is defined solely by the claims attached hereto.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments of the disclosure.

FIG. 1 illustrates a cross-sectional view of an example activity monitoring device that may be used to implement embodiments of the technology disclosed herein.

FIG. 2 illustrates a perspective view of an example activity monitoring device.

FIG. 3 illustrates a cross-sectional view of an example assembled activity monitoring device.

FIG. 4 illustrates a side view of an example electronic capsule.

FIG. 5 illustrates a cross-sectional view of an example electronic capsule.

FIG. 6 illustrates perspective views of example wristbands that may be used in implementations of the disclosed activity monitoring device.

FIG. 7A illustrates an example communications environment in which embodiments of the disclosed technology may be implemented.

FIG. 7B is a block diagram illustrating an example computing device that may be used to implement embodiments of the disclosed technology.

FIG. 7C illustrates modules of an example activity monitoring application that may be used to implement embodiments of the disclosed technology.

FIG. 8 illustrates an activity display that may be associated with an activity display module of the activity monitoring application of FIG. 7C.

FIG. 9 illustrates a sleep display that may be associated with a sleep display module of the activity monitoring application of FIG. 7C.

FIG. 10A illustrates an activity recommendation and fatigue level display that may be associated with an activity recommendation and fatigue level display module of the activity monitoring application of FIG. 7C

FIG. 10B is an operational flow diagram illustrating a particular method of generating the display of FIG. 10A.

FIG. 11 illustrates a biological data and intensity recommendation display that may be associated with a biological data and intensity recommendation display module of the activity monitoring application of FIG. 7C.

FIG. 12 illustrates an example computing module that may be used to implement various features of the systems and methods for estimating sky light probes disclosed herein.

### DETAILED DESCRIPTION

The technology disclosed herein is directed toward systems and methods for interacting with and displaying data from an activity monitoring device. One embodiment of the disclosure includes a graphical user interface that enables a user to navigate between different data displays that graphically depict biological data collected from an activity monitoring device, as well as recommendations for sleep and activity levels.

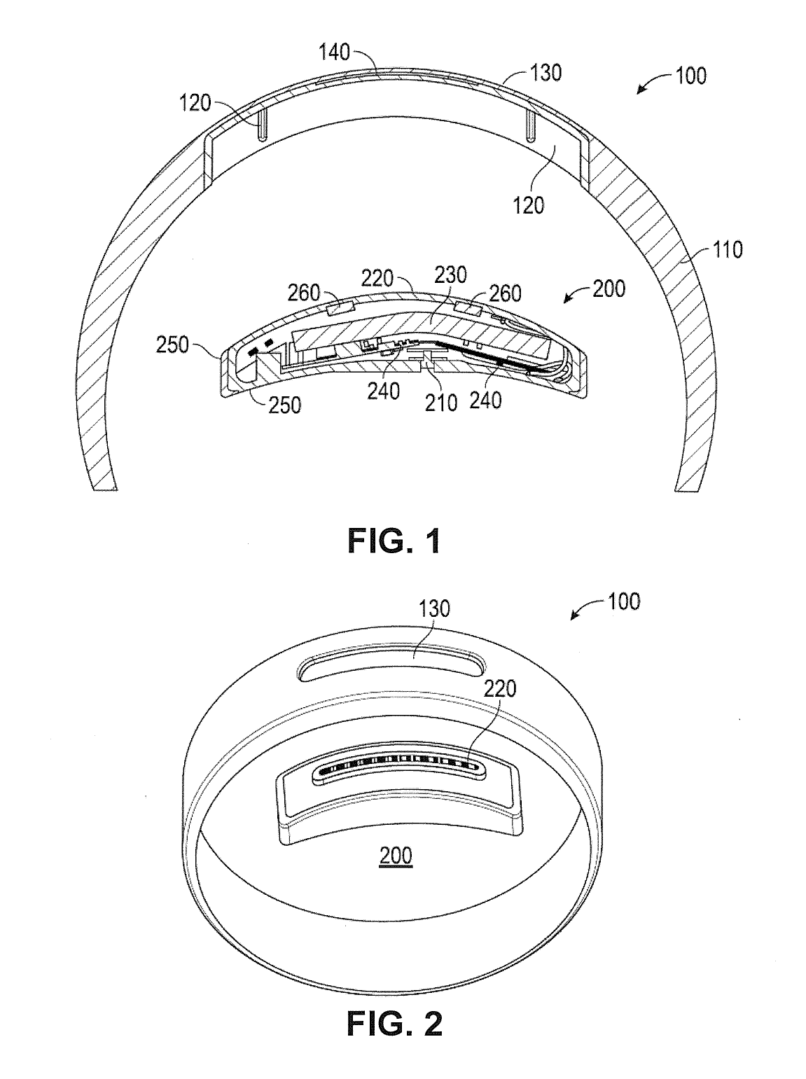

FIG. 1 is a diagram illustrating a cross-sectional view of an exemplary activity monitoring device that may be used to implement the technology disclosed herein. The activity monitoring device comprises an electronic capsule 200 and a wristband 100. The electronic capsule 200 comprises a wrist biosensor 210, a finger biosensor 220, a battery 230, one or more logic circuits 240, and a casing 250. By way of example, the logic circuits may be printed circuit boards (PCB's). Finger biosensor 220 protrudes outward from a first side of casing 250, and wrist biosensor 210 protrudes outward from a second side of casing 250. The battery 230 and logic circuits 240 are enclosed inside of the casing 250. In some embodiments, casing 250 is molded plastic or another moldable material. Additionally, the casing may be sealed using an ultrasonic welding process such that it is watertight. Battery 230 is electronically coupled and supplies power to the logic circuits 240.

In some embodiments, the one or more logic circuits 240 comprise an accelerometer, a wireless transmitter, and circuitry. The logic circuits may further comprise a gyroscope. These logic circuits may be configured to process electronic input signals from the biosensors and the accelerometer, store the processed signals as data, and output the data using the wireless transmitter. The transmitter is configured to communicate using available wireless communications standards. For example, in some embodiments, the wireless transmitter may be a BLUETOOTH transmitter, a Wi-Fi transmitter, a GPS transmitter, a cellular transmitter, or some combination thereof. In an alternative embodiment, the wireless transmitter may further comprise a wired interface (e.g. USB, fiber optic, HDMI, etc.) for communicating stored data.

The logic circuits 240 are electrically coupled to the wrist biosensor 210 and the finger biosensor 220. In addition, the logic circuits are configured to receive and process a plurality of electric signals from each of the wrist biosensor 210 and finger biosensor 220. In some embodiments, the plurality of electric signals comprise an activation time signal and a recovery time signal. In these embodiments, the logic circuits 240 may process the plurality of signals to calculate an activation recovery interval equal to the difference between the activation time signal and the recovery time signal. In some embodiments, the plurality of signals may comprise electro-cardio signals from a heart. In these embodiments, the logic circuits may process the electro-cardio signals to calculate and store a RR-interval. The “RR-interval” may be the delta in time between two R-waves, where the R-waves are the electro-cardio signals generated by a ventricle contraction in the heart. In these embodiments, the RR-interval may be used to calculate and store a heart rate variability (HRV) value that indicates the variation over time between consecutive heartbeats.

In some embodiments, the logic circuits may further detect and store metrics such as the amount of physical activity, sleep, or rest over a period of time, or the amount of time without physical activity over a period of time. The logic circuits may use the HRV, the metrics, or some combination thereof to calculate a recovery score. In various embodiments, the recovery score may indicate the user's physical condition and aptitude for further physical activity for the current day. For example, the logic circuits may detect the amount of physical activity and the amount of sleep a user experienced over the last 48 hours, combine those metrics with the user's HRV, and calculate a recovery score. In various embodiments, the calculated recovery score may be based on any scale or range, such as, for example, a range between 1 and 10, a range between 1 and 100, or a range between 0% and 100%.

In some embodiments, the finger biosensor and wrist biosensor may be replaced or supplemented by a single biosensor. In implementations of these embodiments, the single biosensor is an optical biosensor such as a pulse oximeter configured to detect blood oxygen saturation levels. The pulse oximeter may output a signal to the logic circuits indicating a detected cardiac cycle phase, and the logic circuits may use the cardiac cycle phase data to calculate a HRV value.

Wristband 100 comprises a material 110 configured to encircle a human wrist. In one embodiment, wristband 100 is adjustable. A cavity 120 is notched on the radially inward facing side of the wristband and shaped to substantially the same dimensions as the profile of the electronic capsule. In addition, an aperture 130 is located in the material 110 within cavity 120. The aperture 130 is shaped to substantially the same dimensions as the profile of the finger biosensor 220. The cavity and aperture combination is designed to detachably couple to the electric capsule 200 such that, when the electric capsule 200 is positioned inside cavity 120, the finger biosensor 220 protrudes through the aperture 130. Electronic capsule 200 may further comprise one or more magnets 260 configured to secure capsule 200 to cavity 120. Magnets 260 may be concealed in casing 250. Alternatively, cavity 120 may be configured to conceal magnets 260 when electric capsule 200 detachably couples to the cavity and aperture combination.

Wristband 100 may further comprise a steel strip 140 concealed in material 110 within cavity 120. In this embodiment, when the electronic capsule 200 is positioned within the cavity 120, the one or more magnets 260 are attracted to the steel strip 140 and pull electronic capsule 200 radially outward with respect to the wristband. The force provided by magnets 260 may detachably secure electronic capsule 200 inside cavity 120. In alternative embodiments, the electronic capsule may be positioned inside the wristband cavity and affixed using a form-fit, press-fit, snap-fit, friction-fit, VELCRO, or other temporary adhesion or attachment technology.

FIG. 2 illustrates a perspective view of one embodiment of an activity monitoring device. In this embodiment, wristband 100 and electronic capsule 200 are unassembled. Finger biosensor 220 protrudes from the top of electronic capsule 200, and aperture 130 is substantially the same dimensions as the profile of finger biosensor 220. In one embodiment, material 110 may comprise silicone.

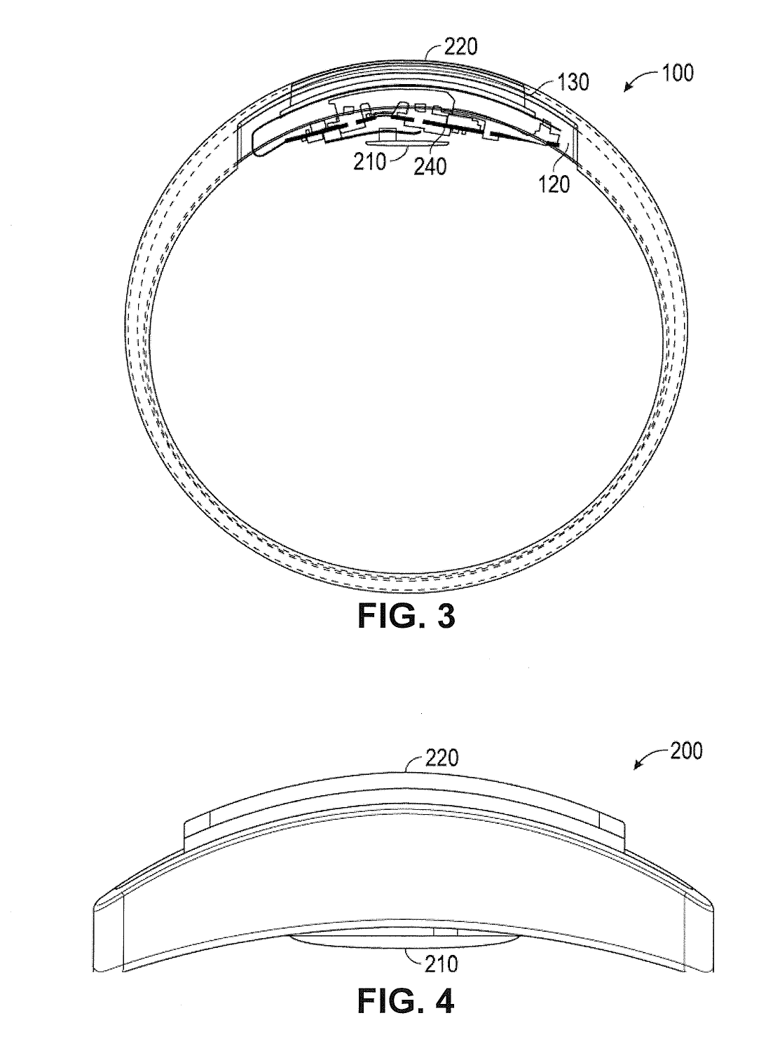

FIG. 3 illustrates a cross-sectional view of one embodiment of a fully assembled wristband with a removable athletic monitoring device. In this embodiment, electronic capsule 200 is positioned inside cavity 120 of wristband 100 such that finger biosensor 220 protrudes through aperture 130. Wrist biosensor 210 protrudes from the radially inward facing side of the assembled device. In this configuration, wrist biosensor 210 may contact the skin on a human wrist when the wrist band is attached to human wrist.

FIG. 4 illustrates a side view of an electronic capsule 200 according to one embodiment of the invention. More particularly, finger biosensor 220 protrudes from a first side of the electronic capsule 200 and wrist biosensor 210 protrudes from a second side of electronic capsule 200. Casing 250 encloses components inside of electronic capsule 200. The casing 250 may comprise moldable plastic. Alternatively, casing 250 may comprise metal, rubber, composite material, or another moldable material. In one embodiment, casing 200 is ultrasonically welded together to make the casing water tight. In alternative embodiments, other methods may be used to make the casing water tight.

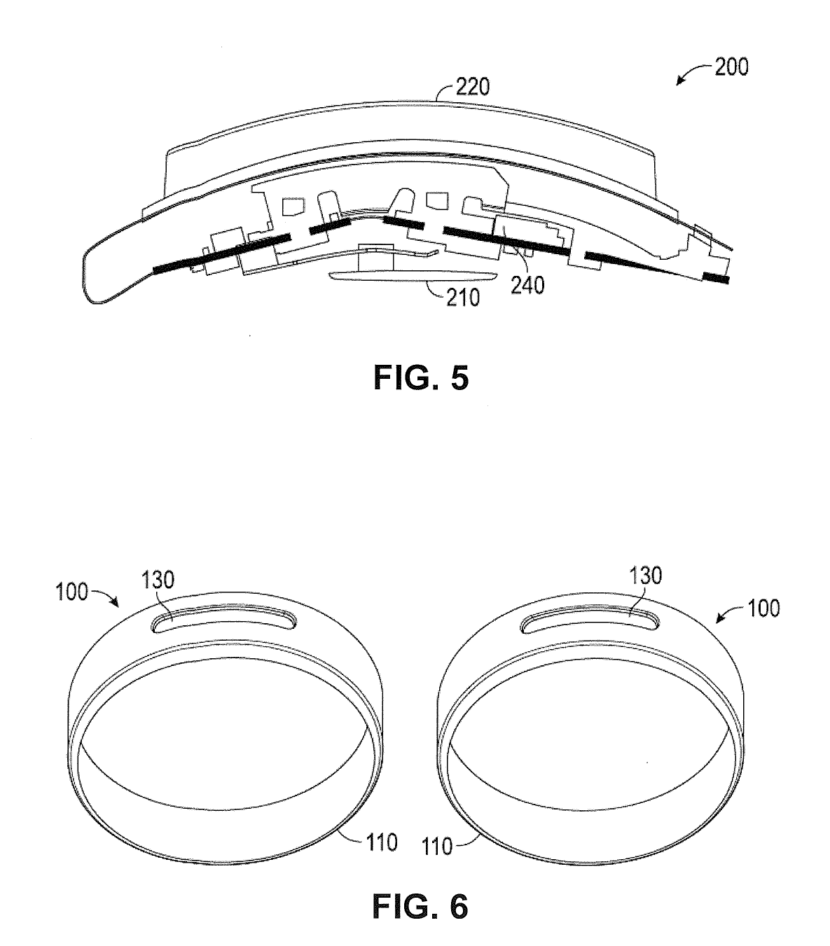

FIG. 5 illustrates a cross-sectional view of electronic capsule 200. In the illustrated embodiment, a finger biosensor 220 protrudes from a first side of the electronic capsule, and a wrist biosensor protrudes from a second side of the electronic capsule. Both the finger biosensor and the wrist biosensor are electronically coupled to the circuit boards 240.

FIG. 6 is a perspective view of two possible variants of a wristband according to embodiments of the technology disclosed herein. Each wristband 100 comprises flexible material 110, and aperture 130 is disposed on each wristband 100. Each electronic capsule may be configured to standard dimensions such that it can be easily removed from one wristband and placed in another wristband. Wristbands may be constructed with different dimensions, including different diameters, widths, and thicknesses, in order to accommodate different human wrist sizes and different preferences. In one embodiment, the wristbands may be adjustable to accommodate different human wrist sizes. Further, the wristbands may be made with different colors, and different flexible materials. In some embodiments, the flexible material 110 comprises silicone. Alternatively, the flexible material may comprise plastic, metal chain links, composite material, leather, synthetic leather, fabric, or other flexible materials.

In some embodiments, the electronic capsule may be detachably coupled to a cavity on a shoe and/or a sock. In other embodiments, the electronic capsule may be detachably coupled to sports equipment. For example, the electronic capsule may be detachably coupled to a skateboard, a bicycle, a helmet, a surfboard, a paddle boat, a body board, a hang glider, or other piece of sports equipment. In these embodiments, the electronic capsule may be affixed to the sports equipment using magnets. Alternatively, in other embodiments, the electronic capsule can be affixed using a form-fit, snap-fit, press-fit, friction-fit suction cup, VELCRO, or other technology.

In one embodiment, the electronic capsule may further comprise an optical sensor such as a heart rate sensor or oximeter. For example, the oximeter may sense HRV by detecting blood oxygenation level changes as changes in coloration at the surface of a user's skin. In this embodiment, the optical sensor may be positioned to face radially inward towards a human wrist when the wristband is fit on the human wrist. Alternatively, the optical sensor may be separate from the electronic capsule, but still detachably coupled to the wristband and electronically coupled to the circuit boards enclosed in the electronic capsule.

In various embodiments, a computing device may receive and display data (e.g., biological information, accelerometer information, gyroscope information, etc.) collected by the activity monitoring activity device, thereby allowing the user to interact with the activity monitoring device and otherwise monitor the user's activity levels, fitness levels, and the like. Further, the computing device may be used to collect activity monitoring data using its own motion sensors (e.g., accelerometers, gyroscope, etc.) Further, still, the computing device may transmit information to the activity monitoring device to configure the activity monitoring device.

In certain embodiments, the computing device may be communicatively coupled to the activity monitoring device using a wireless connection. In other embodiments, the computing device may connect to the Internet and receive activity monitoring data over a web browser. For example, the activity monitoring data collected by the activity monitoring device may be stored on a file server and made accessible via a web server. In implementations of this embodiment, the computing device may remotely receive or send activity monitoring data by accessing the web server. In yet further embodiments, the computing device may be mechanically coupled, electrically coupled, or both mechanically and electrically coupled to the activity monitoring device.

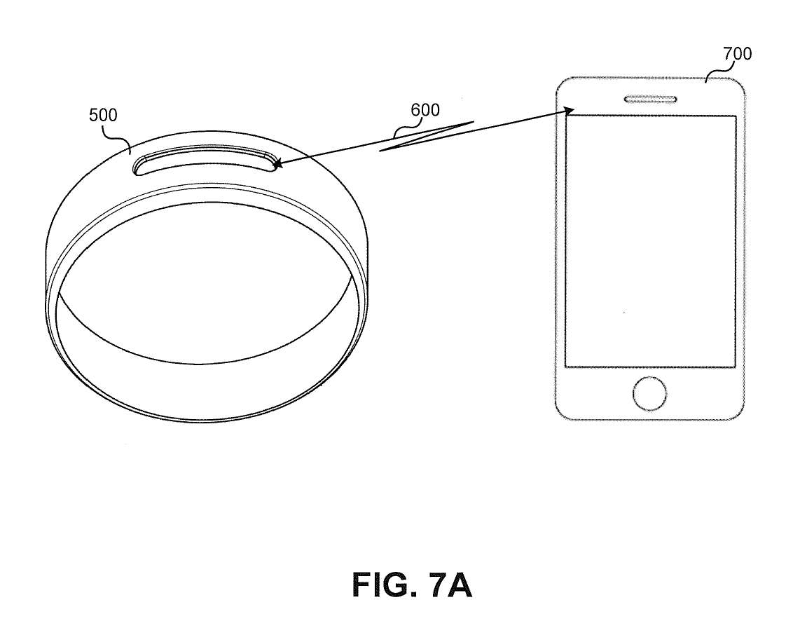

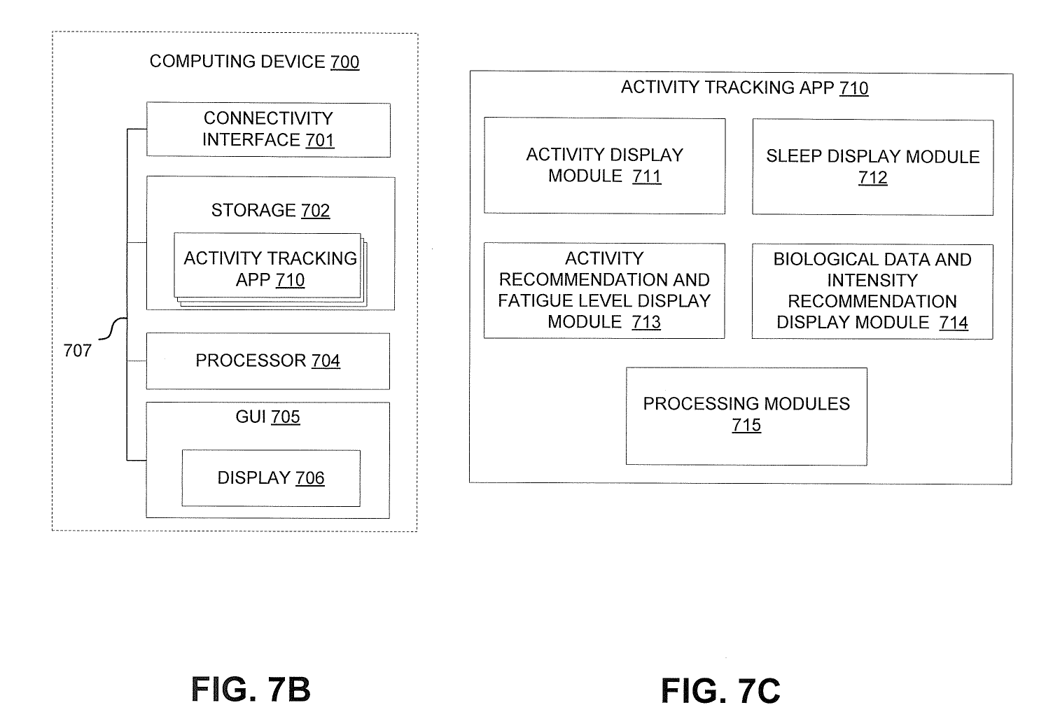

FIG. 7A illustrates an example wireless communications environment in which an activity monitoring device 500 may communicate with a computing device 700 over wireless communication link 600. Computing device 700 may comprise any computing device (smartphone, tablet, laptop, workstation, etc.) configured to receive activity monitoring information from activity monitoring device 500, and provide a graphical user interface (GUI) for displaying and interacting with the data collected by device 500. In additional embodiments, computing device 700 itself may collect additional activity monitoring information that is provided for display. For example, if computing device 700 is a smartphone it may use built in accelerometers and GPS to collect additional data.

The GUI may be provided by various operating systems known in the art, such as, for example, iOS, Android, Windows Mobile, Windows, Mac OS, Chrome OS, Linux, Unix, a gaming platform OS (e.g., Xbox, PlayStation, Wii), etc. In various embodiments, the wireless communication link 600 may be based on one or more wireless communication protocols such as Bluetooth, Zigbee, 802.11 protocols, Infrared (IR), Radio Frequency (RF), etc.

FIG. 7B is a block diagram illustrating example components of computing device 700. As illustrated, computing device 700 comprises a connectivity interface 701, storage 702 with activity tracking application 710, processor 704, a graphical user interface (GUI) 705 including display 706, and a bus 707 for transferring data between the various components of computing device 700.

Connectivity interface 701 connects computing device 700 to activity monitoring device 500 through a communication medium. The medium may comprise a wireless network system such as a Bluetooth system, a Zigbee system, an Infrared (IR) system, a Radio Frequency (RF) system, cellular network, a satellite network, a wireless local area network, or the like. The medium may additionally comprise a wired component such as a USB system.

Storage 702 may comprise volatile memory (e.g. RAM), non-volatile memory (e.g. flash storage), or some combination thereof. In various embodiments, storage 702 may store activity monitoring information collected by activity monitoring device 500. Additionally, storage 702 may store an activity tracking application 710, that when executed by processor 704, allows a user to interact with the collected activity monitoring information.

In various embodiments, a user may interact with activity tracking application 710 via a GUI 705 including a display 706, such as, for example, a touchscreen display that accepts various hand gestures as inputs. In accordance with various embodiments, activity tracking application 710 may process the activity monitoring information collected by activity monitoring device 500 and present it via display 706 of GUI 705. Before describing activity tracking application 710 in further detail, it is worth noting that in some embodiments activity monitoring device 500 may filter the collected activity monitoring information prior to transmitting the information to computing device 700. Accordingly, although the embodiments disclosed herein are described with reference to application 710 processing the received activity monitoring information, in various implementations various preprocessing operations may be performed by a processor of device 500.

In various embodiments, application 710 may be initially configured/setup (e.g., after installation on a smartphone) based on a user's self-reported biological information, sleep information, and activity preference information. For example, during setup a user may be prompted via display 706 for biological information such as the user's gender, height, age, and weight. Further, during setup the user may be prompted for sleep information such as the amount of sleep needed by the user and the user's regular bed time. Further, still, the user may be prompted during setup for a preferred activity level and activities the user desires to be tracked (e.g., running, walking, swimming, biking, etc.) In various embodiments, described below, this self-reported information may be used in tandem with the information collected by device 500 to display activity monitoring information using various modules.

Following setup, activity tracking application 710 may be used by a user to monitor and define how active the user wants to be on a day-to-day basis based on the activity monitoring information (e.g., biological information, accelerometer information, gyroscope information, etc.) collected by activity monitoring device 500. As illustrated, activity tracking application 710 may comprise various display modules, including an activity display module 711, a sleep display module 712, an activity recommendation and fatigue level display module 713, and a biological data and intensity recommendation display module 714. Additionally, activity tracking application 710 may comprise various processing modules 715 for processing the activity monitoring information (e.g., biological information, accelerometer information, gyroscope information, etc.) collected by the activity monitoring device. These modules may be implemented separately or in combination. For example, in some embodiments activity processing modules 715 may be directly integrated with one or more of display modules 711-714.

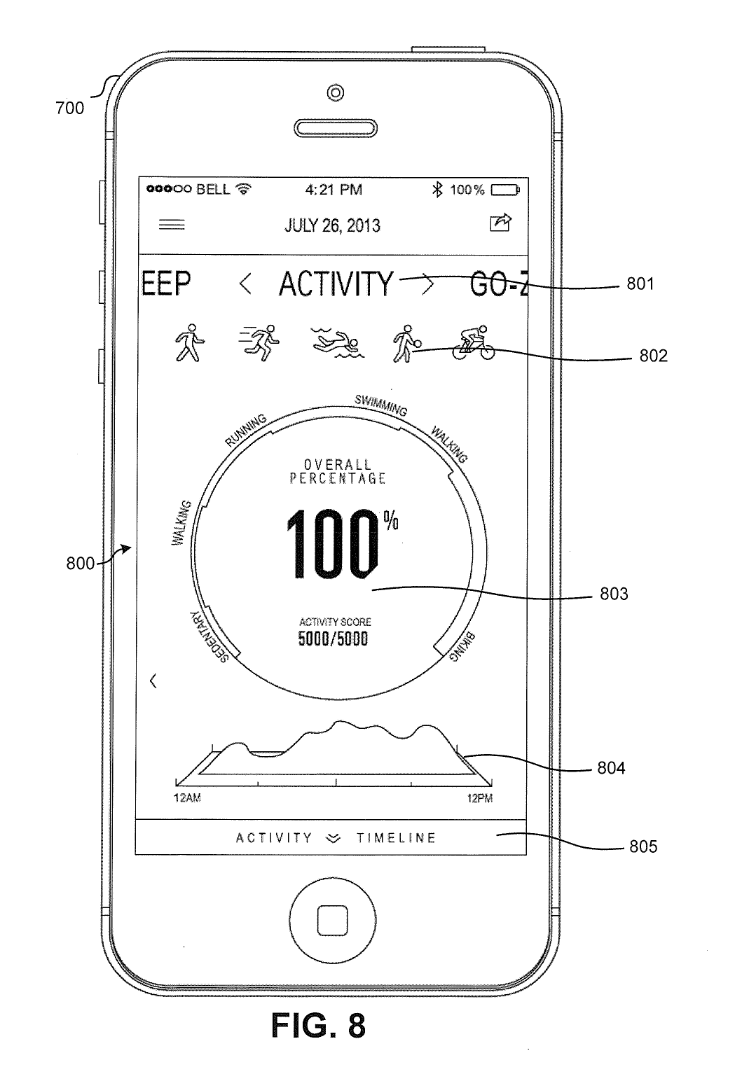

As will be further described below, each of display modules 711-714 may be associated with a unique display provided by activity tracking app 710 via display 706. That is, activity display module 711 may have an associated activity display, sleep display module 712 may have an associated sleep display, activity recommendation and fatigue level display module 713 may have an associated activity recommendation and fatigue level display, and biological data and intensity recommendation display module 714 may have an associated biological data and intensity recommendation display. FIGS. 8-11 illustrate a particular implementation of a GUI for app 710 comprising displays associated with each of display modules 711-714.

FIG. 8 illustrates an activity display 800 that may be associated with an activity display module 711. In various embodiments, activity display 800 may visually present to a user a record of the user's activity. As illustrated, activity display 800 may comprise a display navigation area 801, activity icons 802, activity goal section 803, live activity chart 804, and activity timeline 805. As illustrated in this particular embodiment, display navigation area 801 allows a user to navigate between the various displays associated with modules 711-714 by selecting “right” and “left” arrows depicted at the top of the display on either side of the display screen title. An identification of the selected display may be displayed at the center of the navigation area 801. Other selectable displays may displayed on the left and right sides of navigation area 801. For example, in this embodiment the activity display 800 includes the identification “ACTIVITY” at the center of the navigation area. If the user wishes to navigate to a sleep display in this embodiment, the user may select the left arrow. In implementations where device 700 includes a touch screen display, navigation between the displays may be accomplished via finger swiping gestures. For example, in one embodiment a user may swipe the screen right or left to navigate to a different display screen. In another embodiment, a user may press the left or right arrows to navigate between the various display screens.

In various embodiments, activity icons 802 may be displayed on activity display 800 based on the user's predicted or self-reported activity. For example, in this particular embodiment activity icons 802 are displayed for the activities of walking, running, swimming, sport, and biking, indicating that the user has performed these five activities. In one particular embodiment, one or more modules of application 710 may estimate the activity being performed (e.g., sleeping, walking, running, or swimming) by comparing the data collected by an activity monitoring device's sensors to pre-loaded or learned activity profiles. For example, accelerometer data, gyroscope data, GPS sensor data, biological data, or some combination thereof may be compared to preloaded activity profiles of what the data should look like for a generic user that is running, walking, or swimming. In implementations of this embodiment, the preloaded activity profiles for each particular activity (e.g., sleeping, running, walking, or swimming) may be adjusted over time based on a history of the user's activity, thereby improving the activity predictive capability of the system. In additional implementations, activity display 800 allows a user to manually select the activity being performed (e.g., via touch gestures), thereby enabling the system to accurately adjust an activity profile associated with the user-selected activity. In this way, the system's activity estimating capabilities will improve over time as the system learns how particular activity profiles match an individual user. Particular methods of implementing this activity estimation and activity profile learning capability are described in U.S. patent application Ser. No. 14/568,835, filed Dec. 12, 2014, titled “System and Method for Creating a Dynamic Activity Profile”, and which is incorporated herein by reference in its entirety.

In various embodiments, an activity goal section 803 may display various activity metrics such a percentage activity goal providing an overview of the status of an activity goal for a timeframe (e.g., day or week), an activity score or other smart activity score associated with the goal, and activities for the measured timeframe (e.g., day or week). For example, the display may provide a user with a current activity score for the day versus a target activity score for the day. Particular methods of calculating activity scores are described in U.S. patent application Ser. No. 14/137,734, filed Dec. 20, 2013, titled “System and Method for Providing a Smart Activity Score”, and which is incorporated herein by reference in its entirety.

In various embodiments, the percentage activity goal may be selected by the user (e.g., by a touch tap) to display to the user an amount of a particular activity (e.g., walking or running) needed to complete the activity goal (e.g., reach 100%). In additional embodiments, activities for the timeframe may be individually selected to display metrics of the selected activity such as points, calories, duration, or some combination thereof. For example, in this particular embodiment activity goal section 803 displays that 100% of the activity goal for the day has been accomplished. Further, activity goal section 803 displays that activities of walking, running, biking, and no activity (sedentary) were performed during the day. This is also displayed as a numerical activity score 5000/5000. In this embodiment, a breakdown of metrics for each activity (e.g., activity points, calories, and duration) for the day may be displayed by selecting the activity.

A live activity chart 804 may also display an activity trend of the aforementioned metrics (or other metrics) as a dynamic graph at the bottom of the display. For example, the graph may be used to show when user has been most active during the day (e.g., burning the most calories or otherwise engaged in an activity).

An activity timeline 805 may be displayed as a collapsed bar at the bottom of display 800. In various embodiments, when a user selects activity timeline 805, it may display a more detailed breakdown of daily activity, including, for example, an activity performed at a particular time with associated metrics, total active time for the measuring period, total inactive time for the measuring period, total calories burned for the measuring period, total distance traversed for the measuring period, and other metrics.

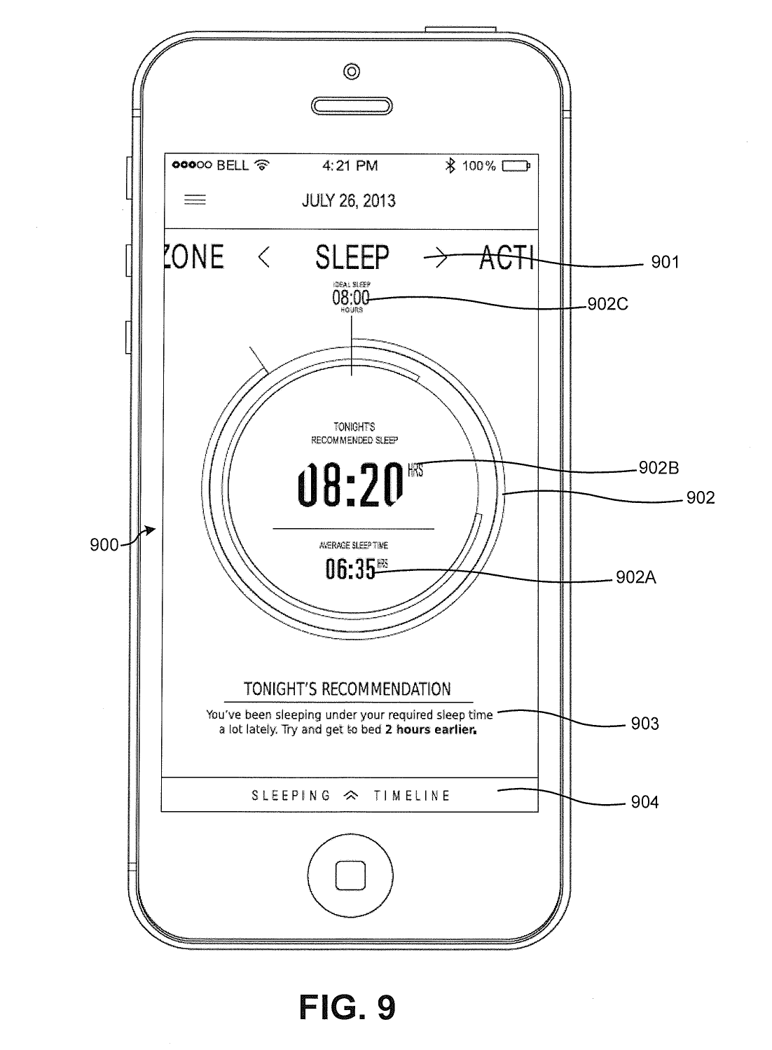

FIG. 9 illustrates a sleep display 900 that may be associated with a sleep display module 712. In various embodiments, sleep display 900 may visually present to a user a record of the user's sleep history and sleep recommendations for the day. It is worth noting that in various embodiments one or more modules of the activity tracking application 710 may automatically determine or estimate when a user is sleeping (and awake) based on an a pre-loaded or learned activity profile for sleep, in accordance with the activity profiles described above. Alternatively, the user may interact with the sleep display 900 or other display to indicate that the current activity is sleep, enabling the system to better learn that individualized activity profile associated with sleep. The modules may also use data collected from the activity monitoring device, including fatigue level and activity score trends, to calculate a recommended amount of sleep. Systems and methods for implementing this functionality are described in greater detail in U.S. patent application Ser. No. 14/568,835, filed Dec. 12, 2014, and titled “System and Method for Creating a Dynamic Activity Profile”, and U.S. patent application Ser. No. 14/137,742, filed Dec. 20, 2013, titled “System and Method for Providing an Interpreted Recovery Score,” both of which are incorporated herein by reference in their entirety.

As illustrated, sleep display 900 may comprise a display navigation area 901, a center sleep display area 902, a textual sleep recommendation 903, and a sleeping detail or timeline 904. Display navigation area 901 allows a user to navigate between the various displays associated with modules 711-714 as described above. In this embodiment the sleep display 900 includes the identification “SLEEP” at the center of the navigation area 901.

Center sleep display area 902 may display sleep metrics such as the user's recent average level of sleep or sleep trend 902A, a recommended amount of sleep for the night 902B, and an ideal average sleep amount 902C. In various embodiments, these sleep metrics may be displayed in units of time (e.g., hours and minutes) or other suitable units. Accordingly, a user may compare a recommended sleep level for the user (e.g., metric 902B) against the user's historical sleep level (e.g., metric 902A). In one embodiment, the sleep metrics 902A-902C may be displayed as a pie chart showing the recommended and historical sleep times in different colors. In another embodiment, sleep metrics 902A-902C may be displayed as a curvilinear graph showing the recommended and historical sleep times as different colored, concentric lines. This particular embodiment is illustrated in example sleep display 900, which illustrates an inner concentric line for recommended sleep metric 902B and an outer concentric line for average sleep metric 902A. In this example, the lines are concentric about a numerical display of the sleep metrics.

In various embodiments, a textual sleep recommendation 903 may be displayed at the bottom or other location of display 900 based on the user's recent sleep history. A sleeping detail or timeline 904 may also be displayed as a collapsed bar at the bottom of sleep display 900. In various embodiments, when a user selects sleeping detail 904, it may display a more detailed breakdown of daily sleep metrics, including, for example, total time slept, bedtime, and wake time. In particular implementations of these embodiments, the user may edit the calculated bedtime and wake time. In additional embodiments, the selected sleeping detail 904 may graphically display a timeline of the user's movements during the sleep hours, thereby providing an indication of how restless or restful the user's sleep is during different times, as well as the user's sleep cycles. For the example, the user's movements may be displayed as a histogram plot charting the frequency and/or intensity of movement during different sleep times.

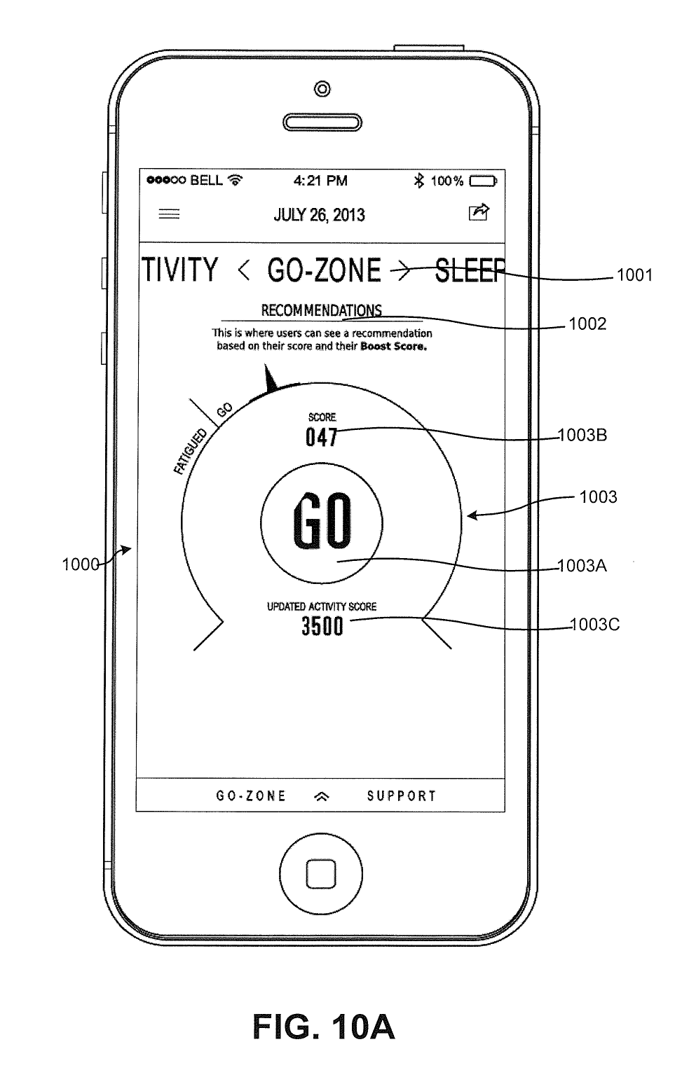

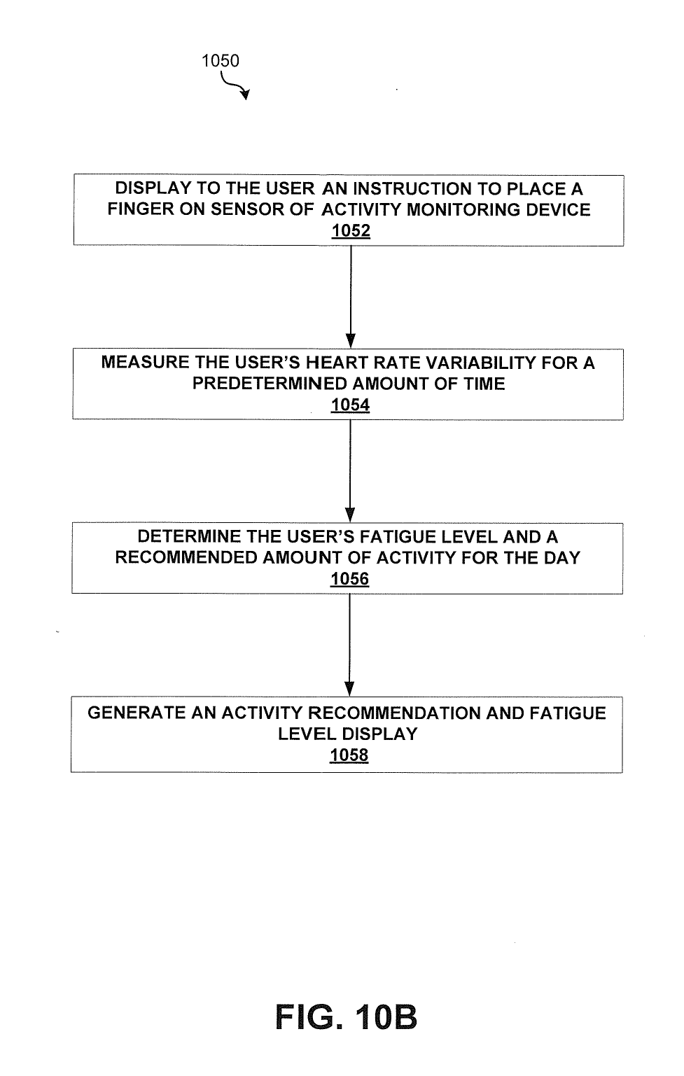

FIG. 10A illustrates an activity recommendation and fatigue level display 1000 that may be associated with an activity recommendation and fatigue level display module 713. In various embodiments, display 1000 may visually present to a user the user's current fatigue level and a recommendation of whether or not engage in activity. It is worth noting that one or more modules of activity tracking application 710 may track fatigue level based on data received from the activity monitoring device 500, and make an activity level recommendation. For example, HRV data tracked at regular intervals may be compared with other biological data to determine how fatigued the user is. Additionally, the HRV data may be compared to pre-loaded or learned fatigue level profiles, as well as a user's specified activity goals. Particular systems and methods for implementing this functionality are described in greater detail in U.S. patent application Ser. No. 14/140,414, filed Dec. 24, 2013, titled “System and Method for Providing an Intelligent Goal Recommendation for Activity Level”, and which is incorporated herein by reference in its entirety.

As illustrated, display 1000 may comprise a display navigation area 1001 (as described above), a textual activity recommendation 1002, and a center fatigue and activity recommendation display 1003. Textual activity recommendation 1002 may, for example, display a recommendation as to whether a user is too fatigued for activity, and thus must rest, or if the user should be active. Center display 1003 may display an indication to a user to be active (or rest) 1003A (e.g., “go”), an overall score 1003B indicating the body's overall readiness for activity, and an activity goal score 1003C indicating an activity goal for the day or other period. In various embodiments, indication 1003A may be displayed as a result of a binary decision—for example, telling the user to be active, or “go”—or on a scaled indicator—for example, a circular dial display showing that a user should be more or less active depending on where a virtual needle is pointing on the dial.

In various embodiments, display 1000 may be generated by measuring the user's HRV at the beginning of the day (e.g., within 30 minutes of waking up.) FIG. 10B is an operational flow diagram illustrating a particular method 1050 of implementing this functionality. At operation 1052, computing device 700 displays to the user an instruction to place a finger on a sensor of activity monitoring device 500. In various embodiments, this instruction may be accompanied by other information, such as, for example, an instruction to remain relaxed while the variability in the user's heart signal (i.e., HRV) is being measured, an amount of time remaining until the HRV has been sufficiently measured, and an indication that the user's HRV is detected. At operation 1054, the user's HRV is measured by monitoring device 500 for a predetermined amount of time (e.g., two minutes). In various embodiments, the amount of remaining HRV measuring time is displayed to the user. Upon completion of HRV measuring (or during HRV measuring), at operation 1056 one or more processing modules of computing device 700 determines the user's fatigue level for the day and a recommended amount of activity for the day. At operation 1058, an activity recommendation and fatigue level display 1000 is generated based on this determination.

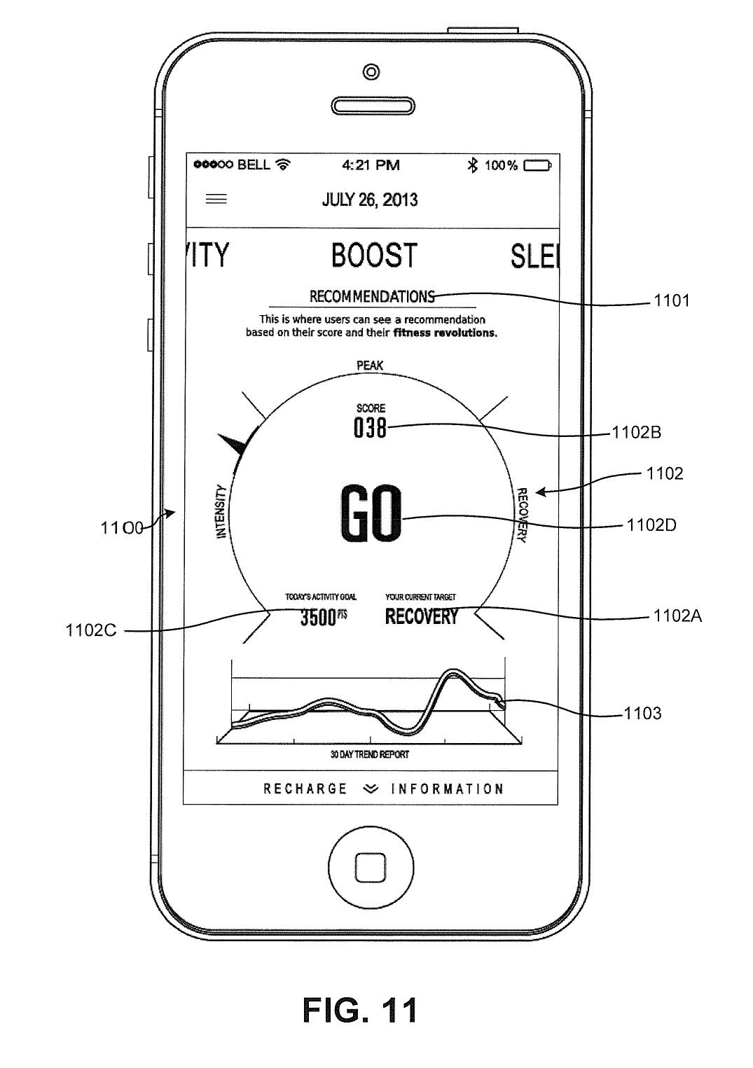

FIG. 11 illustrates a biological data and intensity recommendation display 1100 that may be associated with a biological data and intensity recommendation display module 714. In various embodiments, display 1100 may guide a user of the activity monitoring system through various fitness cycles of high-intensity activity followed by lower-intensity recovery based on the user's body fatigue and recovery level, thereby boosting the user's level of fitness and capacity on each cycle.

As illustrated, display 1100 may include a textual recommendation 1101, a center display 1102, and a historical plot 1103 indicating the user's transition between various fitness cycles. In various embodiments, textual recommendation 1101 may display a current recommended level of activity or training intensity based on current fatigue levels, current activity levels, user goals, pre-loaded profiles, activity scores, smart activity scores, historical trends, and other bio-metrics of interest. Center display 1102 may display a fitness cycle target 1102A (e.g., intensity, peak, fatigue, or recovery), an overall score 1102B indicating the body's overall readiness for activity, an activity goal score 1102C indicating an activity goal for the day or other period, and an indication to a user to be active (or rest) 1102D (e.g., “go”). The data of center display 1102 may be displayed, for example, on a virtual dial, as text, or some combination thereof. In one particular embodiment implementing a dial display, recommended transitions between various fitness cycles (e.g., intensity and recovery) may be indicated by the dial transitioning between predetermined markers.

In various embodiments, display 1100 may display a historical plot 1103 that indicates the user's historical and current transitions between various fitness cycles over a predetermined period of time (e.g., 30 days). The fitness cycles, may include, for example, a fatigue cycle, a performance cycle, and a recovery cycle. Each of these cycles may be associated with a predetermined score range (e.g., overall score 1102B). For example, in one particular implementation a fatigue cycle may be associated with an overall score range of 0 to 33, a performance cycle may be associated with an overall score range of 34 to 66, and a recovery cycle may be associated with an overall score range of 67 to 100. The transitions between the fitness cycles may be demarcated by horizontal lines intersecting the historical plot 1103 at the overall score range boundaries. For example, the illustrated historical plot 1103 includes two horizontal lines intersecting the historical plot. In this example, measurements below the lowest horizontal line indicate a first fitness cycle (e.g., fatigue cycle), measurements between the two horizontal lines indicate a second fitness cycle (e.g., performance cycle), and measurements above the highest horizontal line indicate a third fitness cycle (e.g., recovery cycle).

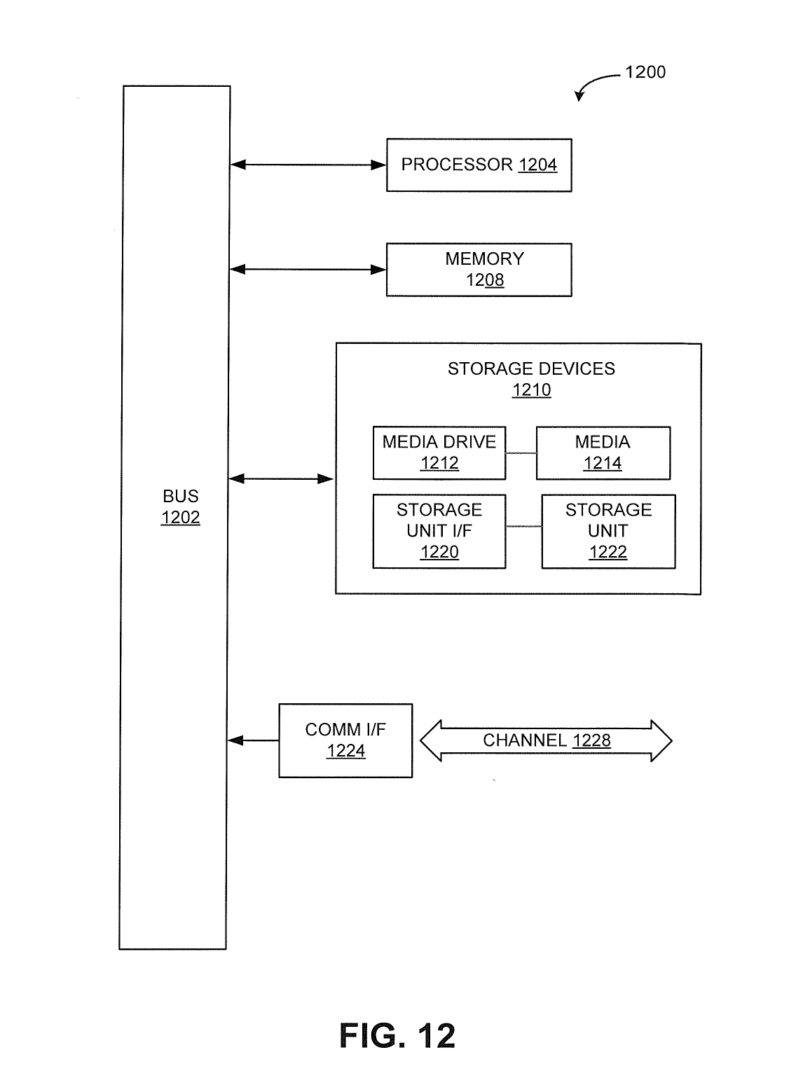

FIG. 12 illustrates an example computing module that may be used to implement various features of the systems and methods for estimating sky probes disclosed herein. As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 12. Various embodiments are described in terms of this example-computing module 1200. After reading this description, it will become apparent to a person skilled in the relevant art how to implement the application using other computing modules or architectures.

Referring now to FIG. 12, computing module 1200 may represent, for example, computing or processing capabilities found within desktop, laptop, notebook, and tablet computers; hand-held computing devices (tablets, PDA's, smart phones, cell phones, palmtops, etc.); mainframes, supercomputers, workstations or servers; or any other type of special-purpose or general-purpose computing devices as may be desirable or appropriate for a given application or environment. Computing module 1200 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 1200 might include, for example, one or more processors, controllers, control modules, or other processing devices, such as a processor 1204. Processor 1204 might be implemented using a general-purpose or special-purpose processing engine such as, for example, a microprocessor, controller, or other control logic. In the illustrated example, processor 1204 is connected to a bus 1202, although any communication medium can be used to facilitate interaction with other components of computing module 1200 or to communicate externally.

Computing module 1200 might also include one or more memory modules, simply referred to herein as main memory 1208. For example, preferably random access memory (RAM) or other dynamic memory, might be used for storing information and instructions to be executed by processor 1204. Main memory 1208 might also be used for storing temporary variables or other intermediate information during execution of instructions to be executed by processor 1204. Computing module 1200 might likewise include a read only memory (“ROM”) or other static storage device coupled to bus 1202 for storing static information and instructions for processor 1204.

The computing module 1200 might also include one or more various forms of information storage mechanism 1210, which might include, for example, a media drive 1212 and a storage unit interface 1220. The media drive 1212 might include a drive or other mechanism to support fixed or removable storage media 1214. For example, a hard disk drive, a solid state drive, a magnetic tape drive, an optical disk drive, a CD, DVD, or Blu-ray drive (R or RW), or other removable or fixed media drive might be provided. Accordingly, storage media 1214 might include, for example, a hard disk, a solid state drive, magnetic tape, cartridge, optical disk, a CD, DVD, Blu-ray or other fixed or removable medium that is read by, written to or accessed by media drive 1212. As these examples illustrate, the storage media 1214 can include a computer usable storage medium having stored therein computer software or data.

In alternative embodiments, information storage mechanism 1210 might include other similar instrumentalities for allowing computer programs or other instructions or data to be loaded into computing module 1200. Such instrumentalities might include, for example, a fixed or removable storage unit 1222 and an interface 1220. Examples of such storage units 1222 and interfaces 1220 can include a program cartridge and cartridge interface, a removable memory (for example, a flash memory or other removable memory module) and memory slot, a PCMCIA slot and card, and other fixed or removable storage units 1222 and interfaces 1220 that allow software and data to be transferred from the storage unit 1222 to computing module 1200.

Computing module 1200 might also include a communications interface 1224. Communications interface 1224 might be used to allow software and data to be transferred between computing module 1200 and external devices. Examples of communications interface 1224 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 1224 might typically be carried on signals, which can be electronic, electromagnetic (which includes optical) or other signals capable of being exchanged by a given communications interface 1224. These signals might be provided to communications interface 1224 via a channel 1228. This channel 1228 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 1208, storage unit 1220, media 1214, and channel 1228. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 1200 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A system, comprising:
an activity monitoring device comprising a plurality of biosensors electrically coupled to a circuit board and configured to measure biological information of a user of the activity monitoring device; and
a computing device communicatively coupled to the activity monitoring device, wherein the computing device comprises:
a display;
one or more processors; and
one or more non-transitory computer-readable mediums operatively coupled to at least one of the one or more processors and having instructions stored thereon that, when executed by at least one of the one or more processors, cause:
at least one of the one or more processors to process the biological information measured by the activity monitoring device; and
the display to display an activity display and a sleep display based on the processed biological information.

2. The system of claim 1, wherein the activity monitoring device comprises an electronic capsule and a wristband.

3. The system of claim 2, wherein the wristband comprises a cavity notched on a radially inward side of the wristband that is shaped to substantially match a profile of the electronic capsule such that the electronic capsule is form-fit in place when positioned in the cavity.

4. The system of claim 1, wherein the activity display comprises at least three of a display navigation area, activity icons, an activity goal section, a live activity chart, and an activity timeline.

5. The system of claim 4, wherein the activity display comprises a display navigation area, activity icons, an activity goal section, a live activity chart, and an activity timeline.

6. The system of claim 1, wherein the sleep display comprises at least two of a display navigation area, a center sleep display area for displaying sleep time metrics, a textual sleep recommendation, and a collapsed sleep detail section.

7. The system of claim 6, wherein the sleep display comprises a display navigation area, a center sleep display area for displaying sleep time metrics, a textual sleep recommendation, and a collapsed sleep detail section.

8. The system of claim 1, wherein the instructions when executed by at least one of the one or more processors, further cause the display to display a recommendation and fatigue level display based on the processed biological information.

9. The system of claim 8, wherein the instructions when executed by at least one of the one or more processors, further cause the display to display a biological data and intensity recommendation display based on the processed biological information.

10. The system of claim 8, wherein the computing device is communicatively coupled to the activity monitoring device via a Bluetooth connection, and wherein the computing device is a mobile device.

11. A method comprising:
collecting biological information of a user using one or more biosensors on an activity monitoring device;
processing the biological information;
displaying on a device display an activity display based on the processed biological information;
displaying on the device display a sleep display based on the processed biological information; and
displaying on the device display a recommendation and fatigue level display based on the processed biological information.

12. The method of claim 11, wherein the displaying operations are performed by a mobile device communicatively coupled to the activity monitoring device.

13. The method of claim 12, wherein one or more processors of the mobile device process the biological information.

14. The method of claim 13, wherein the processing operation and the displaying operations are performed when the one or more processors of the mobile device execute an activity tracking application stored on a non-transitory computer-readable medium of the mobile device that is operatively coupled to the one or more processors.

15. The method of claim 14, wherein displaying the activity display comprises displaying at least three of a display navigation area, activity icons, an activity goal section, a live activity chart, and an activity timeline.

16. The method of claim 14, wherein displaying the sleep display comprises displaying at least three of a display navigation area, a center sleep display area with one or more sleep time metrics, a textual sleep recommendation, and a collapsed sleep detail section.

17. A method, comprising:
displaying to a user an instruction to place a finger on a heart rate sensor of an activity monitoring device;
measuring the user's heart rate variability (HRV) for a predetermined amount of time using the heart rate sensor;
based on the user's measured HRV, determining a fatigue level and recommended amount of activity for the user for a predetermined period; and
displaying to the user the fatigue level and recommended amount of activity for the predetermined period.

18. The method of claim 17, wherein the predetermined period corresponds to a day the user's HRV is measured.

19. The method of claim 18, wherein the displayed instruction, the displayed fatigue level, and the displayed recommend amount of activity are displayed on a mobile device communicatively coupled to the activity monitoring device.

20. The method of claim 19, wherein the activity monitoring device comprises an electronic capsule and a wristband configured to attached to a wrist of the user.
